(19) United States
(12) Patent Application Publication
(10) Pub. No.: US 2012/0253176 A1
(43) Pub. Date: Oct. 4, 2012

(54) Systems and Methods for Debulking Visceral Fat

(75) Inventor: Charles L. Dumoulin, Cincinnati, OH (US)

(73) Assignee: CINCINNATI CHILDREN'S HOSPITAL MEDICAL CENTER, Cincinnati, OH (US)

(21) Appl. No.: 13/509,324

(22) PCT Filed: Nov. 4, 2010
(86) PCT No.: PCT/US10/55351

§ 371 Date: Jun. 14, 2012

Related U.S. Application Data
(60) Provisional application No. 61/260,924, filed on Nov. 13, 2009.

(51) Int. Cl. A61N 7/00; A61B 6/02; A61B 5/055; A61B 8/13; A61B 6/00; A61B 5/01

(52) U.S. Cl. 600/411; 601/2; 600/407; 600/439; 600/427

(57) ABSTRACT

Systems and methods for debulking visceral fat within a subject, include: providing a focused ultrasound transducer configured to focus ultrasonic power at a focal spot; positioning the focused ultrasound transducer with respect to the subject so that the focused ultrasound transducer is enabled to transfer ultrasonic power into the subject; locating the focal spot of the focused ultrasound transducer with respect to at least one target region containing visceral fat within the subject; and debulking visceral fat within the target region by applying ultrasonic energy from the focused ultrasound transducer with sufficient power to cause the death of visceral fat tissue within the target region.

### CROSS REFERENCE TO RELATED APPLICATIONS

The present application claims priority from U.S. Provisional Application, Ser. No. 61/260,924, filed Nov. 13, 2009; the disclosure of which is incorporated herein by reference.

### FIELD OF THE INVENTION

The present disclosure relates generally to the non-invasive reduction of visceral fat in a patient, and more particularly, to systems and methods for using Magnetic Resonance (MR) image guided delivery of focused ultrasound energy into visceral fat for the treatment of disease.

### BACKGROUND OF THE INVENTION

The following description includes information that may be useful in understanding the present subject matter. It is not an admission that any of the information provided herein is prior art or relevant to the presently claimed subject matter, or that any publication specifically or implicitly referenced is prior art.

The role of visceral fat in the development of disease is an area of active research. Endocrine disorders that are characterized by elevated levels of glucocorticoids (GCs), are associated with predominantly abdominal adipose stores, as indicated by high waist to hip ratios and increased intra-abdominal fat deposition. The fact that lipoprotein lipase activity is 2-4 times higher, while the concentration of glucocorticoid receptors is fourfold greater in visceral fat than in subcutaneous fat, may provide an explanation as to how adrenal steroids influence the pattern of adipose tissue deposition. Therefore, a greater magnitude of adrenocorticoid effect is expressed in visceral, than in subcutaneous, fat.

Fontana et. al. (Diabetes 56:1010-1013, 2007) investigated the relationship between visceral fat and its association with noninfectious inflammation. Their studies suggest that visceral fat is an important site for interleukin (IL-6) secretion which is an important mechanistic link between visceral fat and systemic inflammation in people with abdominal obesity. Diamant et. al. (J Clin Endocrinol Metab 90: 1495-1501, 2005) noted that central obesity, insulin resistance, inflammation, as well as vascular changes are common in patients with type 2 diabetes. They also reported that carotid stiffness is associated with visceral obesity in patients with uncomplicated type 2 diabetes, but this association seems to be mediated by circulating IL-6 and C-reactive Protein (CRP), of which IL-6, at least in part, originates from adipose tissue and stimulates hepatic CRP production. Einstein et.al. (Am. J. Phisoi Endocrinol Metab 294:E451-E455, 2008) report that the accretion of visceral fat is an important component in the development of hepatic insulin resistance in pregnancy, and accumulation of hepatic triglycerides is a mechanism by which visceral fat may modulate insulin action in pregnancy.

The role of visceral fat in human disease does not appear to be limited to metabolic syndromes. For example, Thakore et. al. (Biol. Psychiatry 41:1140-1142, 1997) have observed a correlation between visceral fat deposition and depression. Weber-Hamann et. al. (Psychoneuroendocrinology 31:347-354, 2006) reported that depressed patients showed a larger accumulation of visceral fat mass over time compared to normal patients, and that this increased accumulation is independent of the patient's cortisol concentrations. In addition, Shimomura et. al. (Nature Medicine, 2(7):800-803, 1996) reported that plasma plasminogen activator inhibitor-1 (PAI-1) levels were closely correlated with visceral fat area but not with subcutaneous fat area in human subjects. Since PAI-1 is known to play a role in the development of vascular disease, it appears likely that visceral fat may have a role in the development of vascular disease in visceral obesity.

The strong role that visceral fat plays in providing hormones to the body is not in question. Furthermore, there is evidence that peripheral fat does not play a similar role (New England J. Med. 350(25):2549-2557, 2004). Many studies have shown that visceral fat correlates with increased disease states for Type 2 diabetes, gestational diabetes, cardiovascular disease and depression. Furthermore, the resection of visceral fat appears to restore normal endocrine function in gestational diabetes (Am. J. Phisoi Endocrinol Metab 294:E451-E455, 2008). Although evidence that cardiovascular disease and depression could be treated with visceral fat resection is not yet available, it is reasonable to hypothesize that visceral fat is a contributing factor rather than a consequence of those conditions. Consequently, it is not unreasonable to suppose that visceral fat resection could provide a means to treat multiple disease conditions.

In view of the foregoing, it may be understood that the reduction of visceral fat may serve as a therapeutic method for the treatment of multiple disease conditions. Furthermore, it may be understood that minimally-invasive and non-invasive approaches to achieve visceral fat reduction are more desirable than conventional open surgery.

### SUMMARY

Embodiments of the present disclosure provide a minimally-invasive and non-invasive system and/or method for the reduction of visceral fat. In an embodiment, focused ultrasound energy is employed to ablate visceral fat without detriment to nearby tissue. Focused ultrasound provides a means to destroy tissue deep within the body without damaging tissue that is more superficial (and closer to the focused ultrasound transducer) or deeper (i.e. beyond the focal spot).

In an exemplary embodiment, MR imaging is used to visualize target anatomy during focused ultrasound ablation of visceral fat. In this embodiment temperature sensitive MR imaging may be employed. This temperature sensitive imaging can exploit temperature dependence of one or more intrinsic MR parameters including T1 (also called “spin lattice” or “longitudinal relaxation time,” is a biological parameter that is used in MRIs to distinguish between tissue types), polarization and resonance frequency. MR imaging of target anatomy and temperature sensitive MR imaging during treatment may provide: increased confidence in the ability to selectively target visceral fat, greater ability to avoid damaging non-targeted tissues, and the ability to quantify thermal dose to ensure that target tissue is neither under-ablated nor over-ablated.

In another exemplary embodiment, a system for ablating visceral fat within a subject may comprise an MRI unit and one or more focused ultrasound transducers configured to generate sufficient acoustic power to destructively heat a selected portion of visceral fat within the subject's abdomen. The system may further comprise a device controller in communication with the MRI unit and the one or more focused ultrasound transducers. The device controller may be configured to cause the MRI unit to acquire a first temperature-sensitive image of the region of interest, and to cause at least one region of visceral fat within the abdomen to be heated by the ultrasound transducer. The device controller may cause the MRI unit to acquire subsequent temperature-sensitive images of the region of interest during the heating of visceral fat. The device controller may further subtract (or cause the MRI unit to subtract) the first temperature-sensitive imaging from subsequent temperature-sensitive images to create temperature difference images of the region of interest that provide qualitative and/or quantitative measures of tissue temperature in response to the heating of visceral fat, thereby determining efficacy of treatment.

In a first aspect of the disclosure, a method of debulking visceral fat within a subject is provided. The method may include the steps of: providing a focused ultrasound transducer configured to focus ultrasonic power at a focal spot; positioning the focused ultrasound transducer with respect to the subject so that the focused ultrasound transducer is enabled to transfer ultrasonic power into the subject; locating the focal spot of the focused ultrasound transducer with respect to at least one target region containing visceral fat within the subject; and debulking visceral fat within the target region by applying ultrasonic energy from the focused ultrasound transducer with sufficient power to cause the death of visceral fat tissue within the target region.

With respect to this first aspect, the focused ultrasound transducer may be a piezoelectric device. In a more detailed embodiment, the positioning step may include placing a selected material between the focused ultrasound transducer and the subject; where the material has an acoustic impedance substantially similar to that of the subject.

With respect to this first aspect, the focused ultrasound transducer may be a Capacitive Micromachined Ultrasound Transducer (CMUT). With such a transducer, the positioning step may include a step of aiming the CMUT focused ultrasound transducer at the subject through an air gap.

With respect to the first aspect, the locating step may include use of an internal body tissue imaging system. With such an embodiment, the locating step may include using the internal body tissue imaging system to locate a heating effect of the subject's internal body tissue associated with the application of ultrasonic energy at the focal spot. Further, the imaging system may include an MR scanner. With such an imaging system, the imaging system may employ a step of measuring proton-resonance frequency shift during the step of locating a heating effect of the subject's internal body tissue associated with the application of ultrasonic energy at the focal spot. Alternatively, or in addition, the imaging system may employ a step of measuring change of longitudinal relaxation time, T1, during the step of locating a heating effect of the subject's internal body tissue associated with the application of ultrasonic energy at the focal spot. Alternatively, or in addition, the imaging system may employ a step of measuring change in net polarization during the step of locating a heating effect of the subject's internal body tissue associated with the application of ultrasonic energy at the focal spot.

With respect to the first aspect, the locating step may include applying energy from the focused ultrasound transducer at a lower power than during the step of debulking visceral fat. Alternatively, or in addition, the method may further include a step of moving the focal spot of the focused ultrasound transducer with respect to the at least one target region containing visceral fat within the subject after or during the locating step. Such moving step may include physically moving the focused ultrasound transducer with respect to the subject and/or changing a relative amplitude of a drive signal of the focused ultrasound transducer and/or changing a relative phase of the drive signal of focused ultrasound transducer.

With respect to the first aspect, the imaging system includes an ultrasound scanner or the imaging system may include an x-ray scanner.

With respect to the first aspect, the method may also include a step of monitoring application of ultrasonic power during or after the step of debulking visceral fat using an internal body tissue imaging system. This monitoring step may include using the internal body tissue imaging system, such as an MR scanner, to measure a heating effect of the subject's internal body tissue associated with the application of ultrasonic energy at the focal spot during or after the step of debulking visceral fat. With such a monitoring step, the imaging system may employ a step of measuring proton-resonance frequency shift during the step of measuring a heating effect of the subject's internal body tissue associated with the step of debulking visceral fat. For example, the imaging system may employ a step of measuring change of longitudinal relaxation time, T1, during the step of measuring a heating effect of the subject's internal body tissue associated with the step of debulking visceral fat; and/or the imaging system may employ a step of measuring change in net polarization during the step of measuring a heating effect of the subject's internal body tissue associated with the step of debulking visceral fat.

With respect to the first aspect, the positioning step may include a step of acoustically coupling the focused ultrasound transducer with respect to the subject.

With respect to the first aspect, the step of debulking visceral fat within the target region may include a step of slewing the focal spot within the target region. Such a slewing step may include changing the shape of the transducer, employing an acoustic lens and/or applying selected amplitudes and phases to the elements of the multi-element transducer, for example.

With respect to the first aspect, the step of debulking visceral fat within the target region may include applying an ultrasound energy level and at a time duration suitable for debulking visceral fat by thermal necrosis; alternatively, or in addition, the step of debulking visceral fat within the target region may include applying an ultrasound energy level and at a time duration suitable for cavitation.

In a second aspect of the disclosure to provide a system for debulking visceral fat in a subject is provided. Such a system may include: a focused ultrasound transducer configured to deliver sufficient ultrasonic energy to kill tissue within a focal spot; an internal body imaging system configured to locate a heating effect of the subject's internal body tissue associated with the application of ultrasonic energy at the focal spot; an internal body imaging system controller containing appropriate controls and components to operate the internal body imaging system and configured to process the images and other data obtained by the internal imaging system; and a device controller in communication with the focused ultrasound transducer and the internal body imaging system controller; configured to locate the focal spot to a region of visceral fat within the subject.

With this second aspect, examples of the focused ultrasound transducer may include a piezoelectric device or a Capacitive Micromachined Ultrasound Transducer (CMUT).

With this second aspect, the system may further include an acoustic coupling between the ultrasound transducer and the subject. Such an acoustic coupling may include a selected material having an acoustic impedance substantially similar to that of the subject.

With this second aspect, the imaging system may be an MR scanner. With such an embodiment, the imaging system may employ a Proton Resonance Frequency pulse sequence to measure temperature changes. Alternatively, or in addition, the imaging system may employ a pulse sequence sensitive to changes in T1 arising from temperature changes. Alternatively, or in addition, the imaging system may employ a pulse sequence sensitive to changes in polarization arising from temperature changes.

With this second aspect, the imaging system may be an ultrasound scanner or an x-ray scanner, for example.

With this second aspect, the device controller may be configured to control an ultrasound energy level of the ultrasound transducer and time duration suitable for debulking visceral fat by thermal necrosis. Alternatively or in addition, the device controller may be configured to control an ultrasound energy level of the ultrasound transducer and time duration suitable for debulking visceral fat by cavitation.

With this second aspect, the system may employ a focused ultrasound transducer that includes a single piezoelectric crystal. Alternatively, the focused ultrasound transducer may include plurality of elements, each driven with a selected amplitude and phase. With such a focused ultrasound transducer, the device controller may be configured to vary the amplitude and phase of the drive signals used to drive the plurality of elements to move the focal spot without moving the focused ultrasound transducer with respect to the subject.

With this second aspect, the system may further include an actuator configured to move the focal spot during the application of ultrasound power under control of the device controller.

With this second aspect, the device controller may be configured to control the focused ultrasound transducer. Alternatively, or in addition the device controller may be configured to monitor the efficacy of the focused ultrasound transducer. In a more detailed embodiment, the device controller may be configured to cause the internal body imaging system controller to acquire temperature-sensitive images of the region of interest in the subject, before, during, and after the use of the focused ultrasound transducer.

With this second aspect, the internal imaging system may be configured to measure proton-resonance frequency shift during the step of locating a heating effect of the subject's internal body tissue associated with the application of the ultrasonic energy at the focal spot. For example, the internal imaging system may be configured to measure change of longitudinal relaxation time, T1, during the step of locating a heating effect of the subject's internal body tissue associated with the application of the ultrasonic energy at the focal spot. Alternatively, or in addition, the internal imaging system is configured to measure change in net polarization during the step of locating a heating effect of the subject's internal body tissue associated with the application of the ultrasonic energy at the focal spot

Support for claimed inventions will now be described in more detail with reference to exemplary embodiments thereof as shown in the accompanying drawings. While support for the claimed inventions is below with reference to exemplary embodiments, it should be understood that the scope of the claimed inventions is not limited thereto. Those of ordinary skill in the art having access to the teachings herein will recognize additional implementations, modifications, and embodiments, as well as other fields of use, which are within the scope of the claimed inventions as described herein, and with respect to which the claimed inventions may be of significant utility.

### BRIEF DESCRIPTION OF THE DRAWINGS

In order to facilitate a fuller understanding of the present invention, reference is now made to the accompanying drawings, in which like elements are referenced with like numerals. These drawings should not be construed as limiting the present invention, but are intended to be exemplary only.

FIG. 1 shows an exemplary MRI system in or for which the techniques for debulking visceral fat in accordance with the present disclosure may be implemented.

FIG. 2 shows an exemplary focused ultrasound transducer delivering focused ultrasonic energy to a target within the body of a patient in accordance with an embodiment of the present disclosure.

FIG. 3 shows an exemplary axial cross section of the human abdomen in which focused ultrasound is used to ablate visceral fat in accordance with an embodiment of the present disclosure.

FIG. 4 shows a flow chart illustrating an exemplary method for ablating visceral fat in accordance with an embodiment of the present disclosure.

FIG. 5 shows a schematic, block diagram representation of an exemplary MRI system according to an embodiment of the present disclosure.

### DETAILED DESCRIPTION

Embodiments of the present disclosure provide systems and/or methods to apply focused ultrasound, as described below, to debulk visceral fat.

MR imaging of internal body tissues may be used for numerous medical procedures, including diagnosis and surgery. In general terms, MR imaging starts by placing a subject in a relatively uniform, static magnetic field. The static magnetic field causes hydrogen nuclei spins to align and precess about the general direction of the magnetic field. Radio frequency (RF) magnetic field pulses are then superimposed on the static magnetic field to cause some of the aligned spins to alternate between a temporary high-energy non-aligned state and the aligned state, thereby inducing an RF response signal, called the MR echo or MR response signal. It is known that different tissues in the subject produce different MR response signals, and this property can be used to create contrast in an MR image. An RF receiver detects the duration, strength, and source location of the MR response signals, and such data are then processed to generate tomographic or three-dimensional images.

MR imaging can also be used effectively during a medical procedure to assist in locating and guiding medical instruments. For example, a medical procedure can be performed on a patient using medical instruments while the patient is in an MRI scanner. The medical instruments may be for insertion into a patient or they may be used externally but still have a therapeutic or diagnostic effect. For instance, the medical instrument can be an ultrasonic device, which is disposed outside a patient's body and focuses ultrasonic energy to ablate or necrose tissue or other material on or within the patient's body. The MRI scanner preferably produces images at a high rate so that the location of the instrument (or the focus of its effects) relative to the patient may be monitored in real-time (or substantially in real-time). The MRI scanner can be used for both imaging the targeted body tissue and locating the instrument, such that the tissue image and the overlaid instrument image can help track an absolute location of the instrument as well as its location relative to the patient's body tissue.

MR imaging can further provide a non-invasive means of quantitatively monitoring in vivo temperatures. This is particularly useful in the above-mentioned MR-guided focused ultrasound (MRgFUS) treatment or other MR-guided thermal therapy where temperature of a treatment area should be continuously monitored in order to assess the progress of treatment and correct for local differences in heat conduction and energy absorption. The monitoring (e.g., measurement and/or mapping) of temperature with MR imaging is generally referred to as MR thermometry or MR thermal imaging.

Among the various methods available for MR thermometry, proton-resonance frequency (PRF) shift method is often preferred due to its excellent linearity with respect to temperature change, near-independence from tissue type, and good sensitivity. The PRF shift method is based on the phenomenon that the MR resonance frequency of protons in water molecules changes linearly with temperature. Since the frequency change is small, only −0.01 ppm/° C. for bulk water and approximately −0.0096-−0.013 ppm/° C. in tissue, the PRF shift is typically detected with a phase-sensitive imaging method in which the imaging is performed twice: first to acquire a baseline PRF phase image prior to a temperature change and then to acquire a second image after the temperature change, thereby capturing a small phase change that is proportional to the change in temperature.

A phase image, for example, may be computed from an MR image, and a temperature-difference map relative to the baseline image may be obtained by (i) subtracting, on a pixel-by-pixel basis, the phase image corresponding to the baseline from the phase image corresponding to a subsequently obtained MR image, and (ii) converting phase differences into temperature differences based on the PRF temperature dependence.

Unfortunately, not all tissue is well-suited for PRF MR thermometry. For example, tissue whose primary MR signal comes from lipids are known to be poorly suited to PRF temperature monitoring because lipids do not have a resonance frequency that depends on temperature. Fortunately, lipids exhibit other observable MR parameters that do change with temperature These observable parameters include longitudinal relaxation time, T1, and net polarization as defined by the Boltzmann equation:

$$\frac{Number of spins in the excited state}{Number of spins in the ground state} = exp ( \frac{- ( {E}_{excited} - {E}_{ground} )}{kT} ) ) \qquad [ 1 ]$$

where the ratio of spins in the excited to ground state represents the spin polarization, E_{excited} is the energy level of the excited state, E_{ground} is the energy of the ground state, T is temperature and k is the Boltzmann constant.

Additional details of MR thermometry for use with the present disclosure can be found in Peters, et. al., “Ex Vivo Tissue-Type Independence Frequency Shift MR Thermometry (MRM 40:454-459 1988); and Rieke, et. al., “MR Thermometry” (Journal of Magnetic Resonance Imaging 27:376-390 2008).

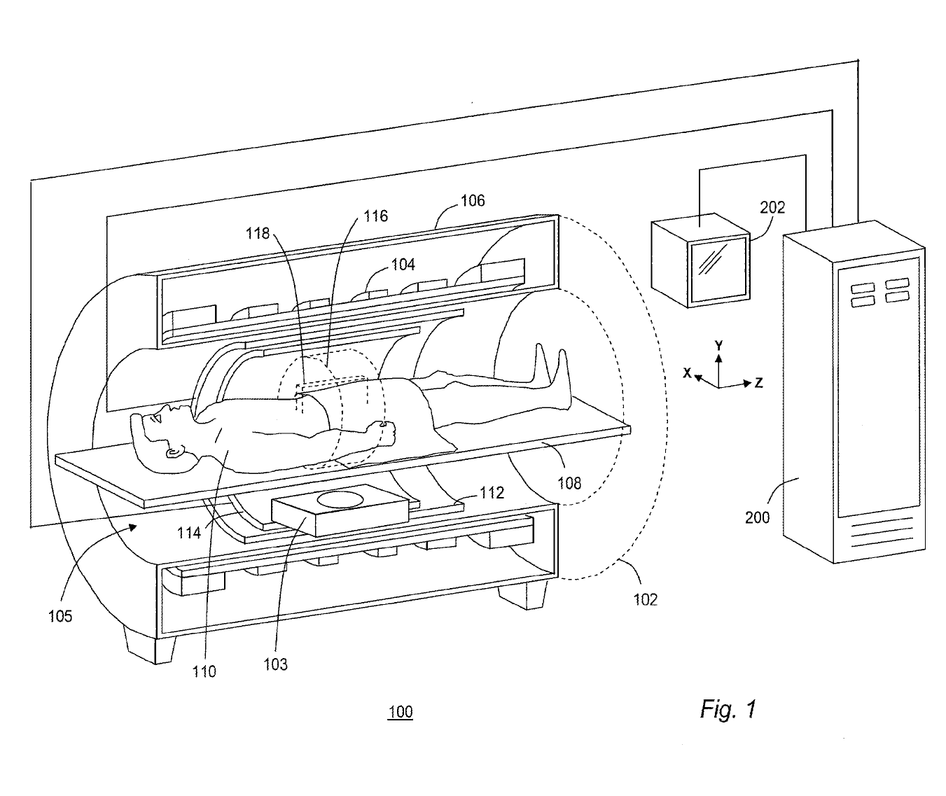

FIG. 1 shows an exemplary MRI system 100 in or for which the techniques for visceral fat debulking in accordance with the present disclosure may be implemented. The illustrated MRI system 100 comprises an MRI scanner 102. If an MR-guided procedure is being performed, a medical device 103 may be disposed within the bore of the MRI scanner 102. Since the components and operation of the MRI scanner are well-known in the art, only some basic components helpful in the understanding of the system 100 and its operation will be described herein.

The MRI scanner 102 typically comprises a cylindrical superconducting magnet 104, which generates a static magnetic field within a bore 105 of the superconducting magnet 104. The superconducting magnet 104 generates a substantially homogeneous magnetic field within an imaging region 116 inside the magnet bore 105. The superconducting magnet 104 may be enclosed in a magnet housing 106. A support table 108, upon which a patient 110 lies, is disposed within the magnet bore 105. A region of interest 118 within the patient 110 may be identified and positioned within the imaging region 116 of the MRI scanner 102.

A set of cylindrical magnetic field gradient coils 112 may also be provided within the magnet bore 105. The gradient coils 112 also surround the patient 110. The gradient coils 112 can generate magnetic field gradients of predetermined magnitudes, at predetermined times, and in three mutually orthogonal directions within the magnet bore 105. With the field gradients, different spatial locations can be associated with different precession frequencies, thereby giving an MR image its spatial resolution. An RF transmitter coil 114 surrounds the imaging region 116 and the region of interest 118. The RF transmitter coil 114 emits RF energy in the form of a magnetic field into the imaging region 116, including into the region of interest 118.

The RF transmitter coil 114 can also receive MR response signals emitted from the region of interest 118. The MR response signals are amplified, conditioned and digitized into raw data using an image processing system 200, as is known by those of ordinary skill in the art. The image processing system 200 further processes the raw data using known computational methods, including fast Fourier transform (FFT), into an array of image data. The image data may then be displayed on a monitor 202, such as a computer CRT, LCD display or other suitable display.

The medical device 103 may also be placed within the imaging region 116 of the MRI scanner 102. In the example shown in FIG. 1, the medical device 103 may be an ultrasonic ablation instrument used for ablating tissue such as fibroids or cancerous (or non-cancerous) tissue, for breaking up occlusion within vessels, or for performing other treatment of tissues on or within the patient 110. In fact, the medical device 103 can be any type of medical instrument including, without limitation, a needle, catheter, guidewire, radiation transmitter, endoscope, laparoscope, or other instrument. In addition, the medical device 103 can be configured either for placement outside the patient 110 or for insertion into the patient body. The medical device 103, or at least the portions/components of the medical device 103 placed within the vicinity of the MRI scanner 102, is made from materials and components suitable for use within the MRI scanner 102. Such materials and/or components may include materials and components with sufficiently low magnetic susceptibilities, as is known in the art.

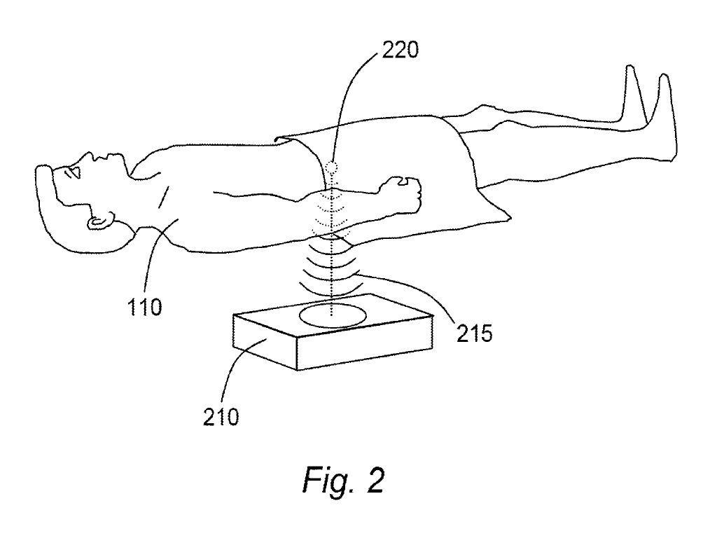

FIG. 2 illustrates one embodiment of a medical device 103. This embodiment is comprised of a focused ultrasound transducer 210 that is capable of creating ultrasonic pressure waves that propagate along a propagation path 215 into the patient 110. The shape of the focused ultrasound transducer 210 is designed to provide a focal spot 220 of ultrasonic energy at a pre-determined or pre-selected distance, or focal length, from the focused ultrasound transducer 210. Focused ultrasound transducer 210 can be comprised of a single piezoelectric element which provides a fixed focal spot 220. Movement of the focal spot 220 with such a single element system can be accomplished by moving the focused ultrasound transducer 210 with respect to the patient 110. In an alternative embodiment of focused ultrasound transducer 210, the transducer is comprised of multiple elements, each driven with a selected amplitude and phase. Movement of the focal spot 220 with such a multi-element system can be accomplished varying the amplitude and phase of the element drive signals. It is worth noting that focused ultrasound transducer 210 can be constructed with piezoelectric elements or with Capacitive Micromachined Ultrasound Transducers (CMUTs).

It is well known to those skilled in the art that matching acoustic impedance along the propagation path 215 minimizes power loss between the focused ultrasound transducer 210 and the focal spot 220. Acoustic impedance matching can be accomplished by providing water (or other similar material having desirable acoustic properties) between focused ultrasound transducer 210 and the surface of patient 110 that is intersected by propagation path 215. CMUTs have the capability to create greater acoustic power than piezoelectric devices with less internal loss, and hence lower internal heating. Consequently, in embodiments of the focused ultrasound transducer 210 employing CMUTs, the acoustic power generated by the focused ultrasound transducer 210 may be sufficient to overcome the power loss between the focused ultrasound transducer 210 and the focal spot 220. In such an approach the use of acoustic matching material inserted between the focused ultrasound transducer 210 and patient 110 may not be necessary.

Additional details pertaining to focused ultrasound transducers for use with according to the present disclosure can be found in Hynynen et. al., “MR Imaging-guided Focused Ultrasound Surgery of Fibroadenomas in the Breast: A Feasibility Study,” Radiology 219:176-185 (April 2001); and Blana, et. al., “High-Intensity Focused Ultrasound for the Treatement of Localized Prostate Cancer: 5-Year Experience,” Urulogy 63:297-300 (2004).

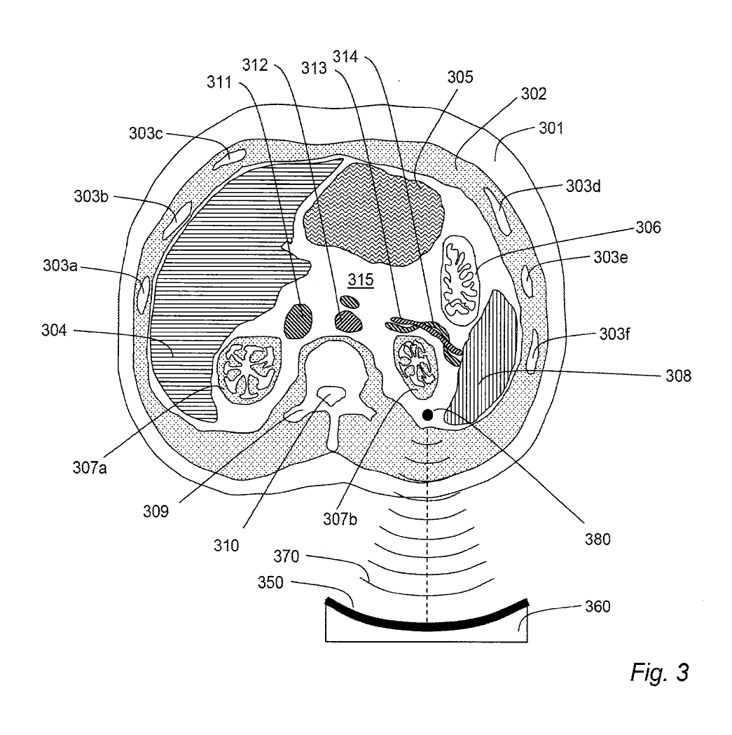

FIG. 3 shows a schematic cross section of the human abdomen. The most superficial layer of tissue that surrounds the abdomen is a wall of peripheral fat 301. Within this wall of peripheral fat 301 is a wall of abdominal muscle 302 which contains a plurality of ribs 303a, 303b, 303c, 303d, 303e and 303f. The largest internal organ in the abdomen is a liver 304. Adjacent to the liver 304 is a stomach 305. Also present in the abdomen is a small intestine 306 that may traverse a given cross section multiple times. The abdomen also contains a right kidney 307a and a left kidney 307b. Other important structures include a spleen 308, vertebra 309, a spinal cord 310, an inferior vena cava 311, an aorta 312, a splenic artery 313, and a splenic vein 314.

Many of the anatomic structures within the wall of abdominal muscle 302 are connected by a matrix of visceral fat 315. This fat surrounds most of the right kidney 307a and left kidney 307b. The matrix of visceral fat 315 can also connect the small intestine 306 and other structures.

FIG. 3 shows the disposition of a medical device 103 in the form of a parabolic focused ultrasound transducer with respect to the abdominal cross section. This parabolic focused ultrasound transducer is comprised of a transducer 350 mounted in a transducer housing 360. Transducer 350 is positioned to provide ultrasonic energy along a sonication path 370 that is focused to a therapeutic hot-spot 380. Sonication path 370 is chosen to avoid bony structures such as vertebra 308 and ribs 303a, 303b, 303c, 303d, 303e, 303f, to minimize power loss within the body due to acoustic impedance mismatch. In accordance with one exemplary embodiment of the present disclosure therapeutic hot-spot 380 is located in the matrix of visceral fat 315. In accordance with another exemplary embodiment of the present disclosure the purpose of positioning the therapeutic hot-spot 380 in visceral fat 315 is to treat disease. Disease conditions that can be treated in this way include, but are not limited to: type 2 diabetes, gestational diabetes, depression and arthrosclerosis.

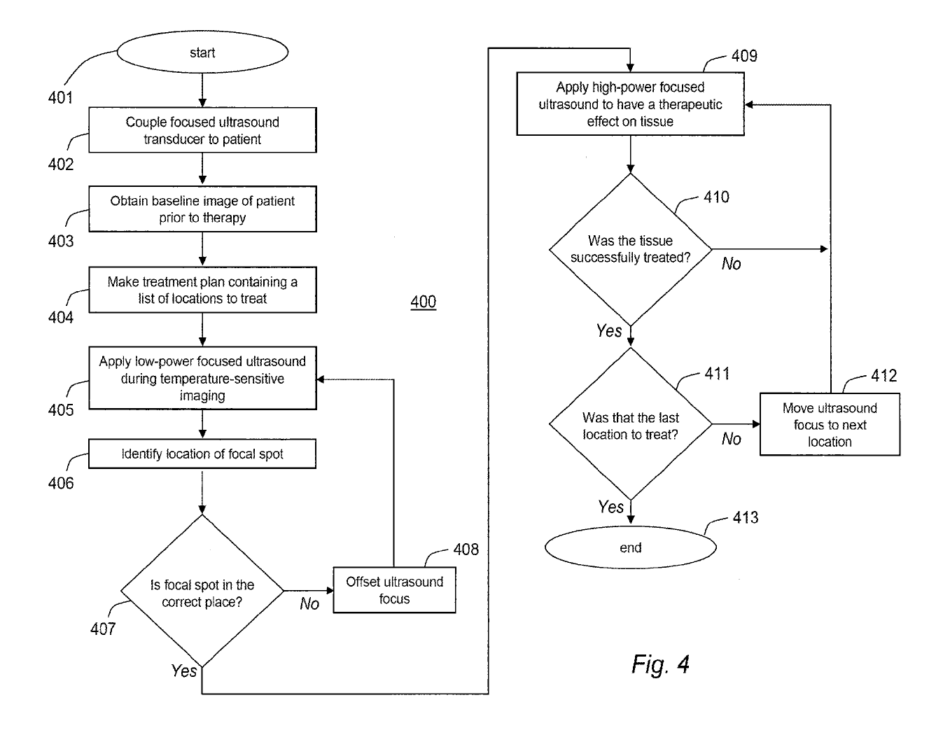

FIG. 4 shows a flow chart 400 illustrating an exemplary method for debulking visceral fat in accordance with an embodiment of the present invention. In step 401, a subject matter such as a human body, may be positioned for treatment. Step 401 may further include the placement of the subject matter in or near an imaging scanner. The imaging scanner may be a magnetic resonance imaging system, ultrasound imaging system or X-ray system. In step 402 a focused ultrasound transducer is acoustically coupled to the subject matter. This acoustic coupling may include the insertion of material whose acoustic impedance substantially matches that of the subject matter, or an air-gap may be left between the transducer and the subject matter if the transducer is sufficiently powerful. In step 403 baseline images of the subject matter are acquired with the imaging scanner. These images may be used to position the subject matter with respect to the focused ultrasound transducer. They may also be used as reference images for computing temperature changes during debulking. In step 404 a treatment plan is made. This plan may employ the baseline images to identify regions containing visceral fat, or be made entirely from external references on the subject matter. In step 405 low-power focused ultrasound energy may be applied during the acquisition of images to test the location of the focal spot. One particularly useful imaging means is temperature-sensitive MR imaging which can be used to visualize the focal spot within the subject matter. The location of the focal spot is determined in step 406. This may involve sensing a distinct temperature change at the focal spot, using temperature-sensitive MR, as compared to the baseline images or related baseline data, for example. If desired, the location of the focal spot may also be determined with Acoustic Radiation Force Imaging using MR in a fashion well-known to those skilled in the art.

In step 407 an evaluation is made to determine if the low-power test focal spot is in the desired location. If the test focal spot is not at the desired location, the relative offset of the test focal spot with respect to the desired focal spot location is measured and used to offset future focal spot generation in step 408. Control of the flow diagram reverts to step 405 and the low-power test is repeated until the focal spot is in the desired location. Offsets of the focal spot pursuant to step 408 can be made by physically moving the transducer with respect to the subject matter and/or vice-versa and/or changing the relative amplitude and/or phases of the drive signals of a multi-element focused ultrasound transducer. In step 407, the power of the ultrasound transducer is lower so that the ablation threshold is not reached while locating the focal spot to the desired location. Generally, it may be desired to keep the temperature rise for this step to be less than 4° C. The power needed to achieve this depends upon the size of the transducer and its frequency. For example, the ultrasound transducer power during the locating step 407 may be generally between 10% and 75% of the power required during the ablation step.

Once the focal spot offset has been measured, applied and verified, the power applied to the transducer is increased to a level and for a period of time sufficient to have a therapeutic effect on the targeted tissue (step 409). For this step, power levels may be selected to be between 1 to 40 Watts (bigger transducers and deeper targets require more power). Frequency may be set at between 1 to 10 MHz (deeper targets may require lower frequencies). Focal spot size may be between 5-25 mm long by 1-5 mm wide. Shot duration may be set from 1 to 10 seconds (higher power generally requires less time to achieve therapeutic temperatures).

Once a target spot has been treated (and/or during the treatment step 409), step 410 is performed in which an evaluation may be made to determine the efficacy of the treatment. This evaluation may include the measurement of reflected power from the ultrasound transducer and/or the assessment of temperature-sensitive images from the imaging scanner. If the evaluation performed in step 410 indicates that the therapeutic effect was not achieved, flow control reverts to step 409. The control loop defined by steps 409 and 410 may be repeated multiple times during the application of power to the target to ensure that a target is not over treated.

Once the evaluation of step 410 indicates that the target tissue has been successfully treated, step 411 is performed to determine if there are more locations in the treatment plan determined in step 404 that need to be targeted. If there are more target locations left to treat, the focal spot is moved in step 412 and flow control reverts to step 409. If there are no more targets to treat, flow control moves to step 413 which terminates the procedure.

The flow chart shown in FIG. 4 may be used with the exemplary apparatus shown in FIG. 1. In this embodiment of the present invention the visceral fat of patient 110 is the targeted tissue. The region of interest 118 in the patient 110 may be identified for purposes of MR temperature measurement using MR thermal imaging or temperature mapping. For example, the region of interest may be a portion of the patient's abdomen as shown in FIG. 1. In general, it is desirable to make the region of interest 118 larger than the focal spot to permit the localization of the focal spot and the subsequent computation of focal spot offsets.

Referring back to FIG. 4, the baseline image obtained in step 403 may be a conventional MR image that provides sufficient contrast to visualize the visceral fat and differentiate it from nearby organs such as the liver, kidneys and spleen. It may also be a phase sensitive image used as a reference for PRF imaging.

Step 406 in FIG. 4 may be performed using a variety of imaging techniques. In one exemplary embodiment PRF imaging is used to reveal temperature changes in tissue associated with the delivery of the low-power focused ultrasound in FIG. 405. In other embodiments changes in tissue parameters such as polarization or T1 can be used to reveal the location of the focal spot. Likewise, the evaluation of treatment success in step 410 may be performed using a PRF imaging, or temperature-sensitive imaging depending on polarization or T1 changes in tissue.

The application of high-power focused ultrasound to tissue such as visceral fat creates local heating. With a sufficient temperature rise and duration of heating, adipose cells within the visceral fat will be killed and no longer be metabolically active. Lipids released by adipose cell death will be either sequestered or released into the rest of the body where they will be either eliminated or reabsorbed by other tissue. Lipids absorbed by untreated visceral fat will not contribute to visceral fat debulking. However, in most subjects the amount of peripheral fat greatly exceeds visceral fat, and thus, any lipids that are reabsorbed in adipose tissue will be reabsorbed primarily in peripheral, rather than visceral fat. Although the ablation of visceral fat will cause immediate cell death, the debulking of visceral fat with focused ultrasound may occur relatively slowly over time. While the reduction of visceral fat volume may be important, it is the elimination of metabolic cell products such as IL-6 and the consequential decrease in C-reactive Protein (CRP) that are likely to have the greatest impact in the treatment of disease.

It should be noted that focused ultrasound can be applied with sufficiently high-power to cause cavitation in tissue. In such an application focused ultrasound can be applied with higher instantaneous power, but lower overall energy. With cavitation, cell death does not necessarily occur due to thermal necrosis. Nevertheless, the end-result of cell death and tissue debulking remains substantially the same.

It should also be noted that more rapid treatments may be possible by defocusing the ultrasound focal spot so that larger volumes are treated in a selected time frame. Defocusing can be accomplished by rapidly slewing the focus over a selected trajectory within the tissue, by changing the shape of the transducer, by employing an acoustic lens, or by applying selected amplitudes and phases to the elements of a multi-element transducer.

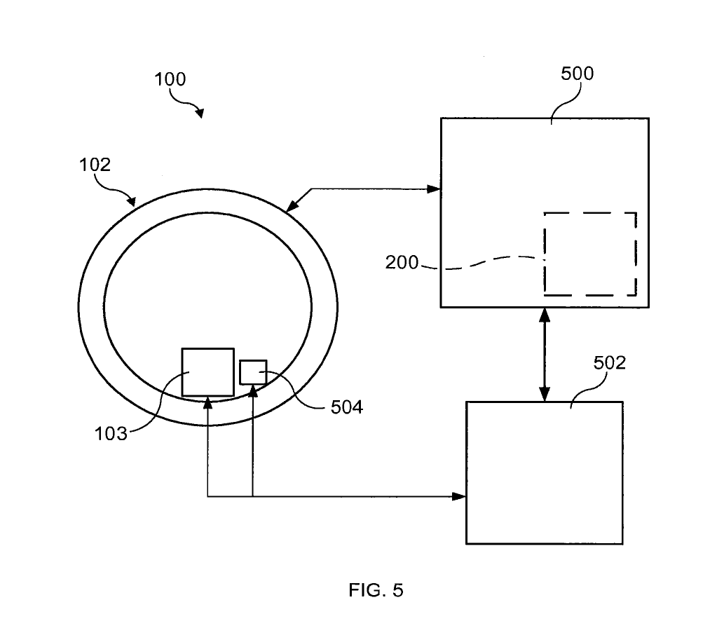

Referring to FIG. 5, the MRI system 100 may include a device controller 502 in communication with the MRI unit's controller 500 (which may include image processing system 200) and in communication with the focused ultrasound transducer 103. MRI unit's controller 500 is any standard controller as known in the art and including the appropriate controls and components to operate the MRI scanner 102 and to process the images and other data obtained therefrom. The device controller 502 may be configured to communicate with the MRI unit controller 500 and the focused ultrasound transducer 103 to locate the focal spot to the desired region of interest in the subject according to the steps discussed above, for example. During this locating process, device controller 502 may also be in communication with optional actuator 504 to control the movement of the ultrasound transducer 103 with respect to the subject. If used, such an actuator 504 could be any form of mechanical device or assembly, electro-mechanical device or assembly, and the like as known to those of ordinary skill sufficient to impart movement of the ultrasound transducer and/or subject with respect to one another under control of the device controller 502. As also discussed herein, the device controller 502 may move the focal spot of the ultrasound transducer 103 with respect to the subject by controlling the ultrasound transducer 103 change a relative amplitude of a drive signal of the focused ultrasound transducer 103 and/or to change a relative phase of the drive signal of focused ultrasound transducer 103.

The device controller 502 may also be configured to control the ultrasound transducer 103 to perform the debulking treatments described herein and to monitor the efficacy of the treatment during or after the treatment. To monitor the efficacy, the device controller 502 may be configured to cause the MRI controller 500 to acquire a first temperature-sensitive image of the region of interest from the subject, and to cause the ultrasound transducer 103 to heat at least one region of visceral fat within the subject's abdomen according to the embodiments disclosed herein. The device controller 502 may cause the MRI unit's controller 500 to acquire subsequent temperature-sensitive images of the region of interest during the heating of visceral fat. The device controller 502 may further subtract (or cause the MRI unit's controller to subtract) the first temperature-sensitive imaging from subsequent temperature-sensitive images to create temperature difference images of the region of interest that provide qualitative and/or quantitative measures of tissue temperature in response to the heating of visceral fat. Such measures may allow the device controller 502 to determine efficacy of treatment, which can lead to the device controller 502 to control the ultrasonic transducer to continue to heat the visceral fat in that region or to stop the therapy in that region.

To provide additional context for various aspects of the present invention, including the device controller 502, the following discussion is intended to provide a brief, general description of a suitable computing environment in which the various aspects of the invention may be implemented, such as any of the processing steps discussed herein. While one embodiment of the device controller 502 relates to the general context of computer-executable instructions that may run on one or more computers, those skilled in the art will recognize that the device controller 502 also may be implemented in combination with other program modules and/or as a combination of hardware and software. For example, and without limitation, the device controller 502 may be embodied in the form of a separate computer system or in the form of a software module resident within the MRI controller 500.

Generally, program modules include routines, programs, components, data structures, etc., that perform particular tasks or implement particular abstract data types. Moreover, those skilled in the art will appreciate that aspects of the inventive methods may be practiced with other computer system configurations, including single-processor or multiprocessor computer systems, minicomputers, mainframe computers, as well as personal computers, hand-held wireless computing devices, microprocessor-based or programmable consumer electronics, and the like, each of which can be operatively coupled to one or more associated devices. Aspects of the device controller 502 may also be practiced in distributed computing environments where certain tasks are performed by remote processing devices that are linked through a communications network. In a distributed computing environment, program modules may be located in both local and remote memory storage devices.

A computer may include a variety of computer readable media. Computer readable media may be any available media that can be accessed by the computer and includes both volatile and nonvolatile media, removable and non-removable media. By way of example, and not limitation, computer readable media may comprise computer storage media and communication media. Non-transitory computer storage media includes volatile and nonvolatile, removable and non-removable media implemented in any method or technology for storage of information such as computer readable instructions, data structures, program modules or other data. Non-transitory computer storage media includes, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which may be used to store the desired information and which may be accessed by the computer.

An exemplary environment for implementing various aspects of the device controller 502 may include a computer that includes a processing unit, a system memory and a system bus. The system bus couples system components including, but not limited to, the system memory to the processing unit. The processing unit may be any of various commercially available processors. Dual microprocessors and other multi processor architectures may also be employed as the processing unit.

The system bus may be any of several types of bus structure that may further interconnect to a memory bus (with or without a memory controller), a peripheral bus, and a local bus using any of a variety of commercially available bus architectures. The system memory may include read only memory (ROM) and/or random access memory (RAM). A basic input/output system (BIOS) is stored in a non-volatile memory such as ROM, EPROM, EEPROM, which BIOS contains the basic routines that help to transfer information between elements within the computer, such as during start-up. The RAM may also include a high-speed RAM such as static RAM for caching data.

The computer may further include an internal hard disk drive (HDD) (e.g., EIDE, SATA), which internal hard disk drive may also be configured for external use in a suitable chassis, a magnetic floppy disk drive (FDD), (e.g., to read from or write to a removable diskette) and an optical disk drive, (e.g., reading a CD-ROM disk or, to read from or write to other high capacity optical media such as the DVD). The hard disk drive, magnetic disk drive and optical disk drive may be connected to the system bus by a hard disk drive interface, a magnetic disk drive interface and an optical drive interface, respectively. The interface for external drive implementations includes at least one or both of Universal Serial Bus (USB) and IEEE 1394 interface technologies.

The drives and their associated computer-readable media may provide nonvolatile storage of data, data structures, computer-executable instructions, and so forth. For the computer, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the invention.

A number of program modules may be stored in the drives and RAM, including an operating system, one or more application programs, other program modules and program data. All or portions of the operating system, applications, modules, and/or data may also be cached in the RAM. It is appreciated that the device controller 502 may be implemented with various commercially available operating systems or combinations of operating systems.

It is within the scope of the disclosure that a user may enter commands and information into the computer through one or more wired/wireless input devices, for example, a touch screen display, a keyboard and/or a pointing device, such as a mouse. Other input devices may include a microphone (functioning in association with appropriate language processing/recognition software as know to those of ordinary skill in the technology), an IR remote control, a joystick, a game pad, a stylus pen, or the like. These and other input devices are often connected to the processing unit through an input device interface that is coupled to the system bus, but may be connected by other interfaces, such as a parallel port, an IEEE 1394 serial port, a game port, a USB port, an IR interface, etc.

A display monitor or other type of display device may also be connected to the system bus via an interface, such as a video adapter. In addition to the monitor, a computer may include other peripheral output devices, such as speakers, printers, etc.

The computer may operate in a networked environment using logical connections via wired and/or wireless communications to one or more remote computers. The remote computer(s) may be a workstation, a server computer, a router, a personal computer, a portable computer, a personal digital assistant, a cellular device, a microprocessor-based entertainment appliance, a peer device or other common network node, and may include many or all of the elements described relative to the computer. The logical connections depicted include wired/wireless connectivity to a local area network (LAN) and/or larger networks, for example, a wide area network (WAN). Such LAN and WAN networking environments are commonplace in offices, and companies, and facilitate enterprise-wide computer networks, such as intranets, all of which may connect to a global communications network such as the Internet.

The computer may be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi (such as IEEE 802.11x (a, b, g, n, etc.)) and Bluetooth™ wireless technologies. Thus, the communication may be a predefined structure as with a conventional network or simply an ad hoc communication between at least two devices.

The controller 502 may also comprise one or more server(s). The server(s) may also be hardware and/or software (e.g., threads, processes, computing devices). The servers may house threads to perform transformations by employing aspects of the disclosure, for example. One possible communication between a client and a server may be in the form of a data packet adapted to be transmitted between two or more computer processes. The data packet may include a cookie and/or associated contextual information, for example. The system may include a communication framework (e.g., a global communication network such as the Internet) that may be employed to facilitate communications between the client(s) and the server(s).

While the foregoing disclosure includes many details and specificities, it is to be understood that these have been included for purposes of explanation and example only, and are not to be interpreted as limitations of the inventions described herein. It will be apparent to those skilled in the art that other modifications to the embodiments described above can be made without departing from the spirit and scope of the inventions as claimed. Accordingly, such modifications are to be considered within the scope of such inventions. Likewise, it is to be understood that it is not necessary to meet any or all of the identified advantages or objects of any of the inventions described herein in order to fall within the scope of the claims, since inherent and/or unforeseen advantages of such inventions may exist even though they may not have been explicitly discussed herein.

All publications, articles, patents and patent applications cited herein are incorporated into the present disclosure by reference to the same extent as if each individual publication, article, patent application, or patent was specifically and individually indicated to be incorporated by reference.

What is claimed is:

1. A method of debulking visceral fat within a subject, the method comprising the steps of:
providing a focused ultrasound transducer configured to focus ultrasonic power at a focal spot;
positioning the focused ultrasound transducer with respect to the subject so that the focused ultrasound transducer is enabled to transfer ultrasonic power into the subject;
locating the focal spot of the focused ultrasound transducer with respect to at least one target region containing visceral fat within the subject; and
debulking visceral fat within the target region by applying ultrasonic energy from the focused ultrasound transducer with sufficient power to cause the death of visceral fat tissue within the target region.

2. The method of claim 1, wherein the focused ultrasound transducer is a piezoelectric device.

3. The method of claim 2, wherein the positioning step includes placing a selected material between the focused ultrasound transducer and the subject; the material having an acoustic impedance substantially similar to that of the subject.

4. The method of claim 1, wherein the focused ultrasound transducer is a Capacitive Micromachined Ultrasound Transducer (CMUT).

5. The method of claim 4, wherein the positioning step includes a step of aiming the CMUT focused ultrasound transducer at the subject through an air gap.

6. The method of claim 1, wherein the locating step includes use of an internal body tissue imaging system.

7. The method of claim 6, wherein the locating step includes using the internal body tissue imaging system to locate a heating effect of the subject's internal body tissue associated with the application of ultrasonic energy at the focal spot.

8. The method of claims 6, wherein the imaging system includes an MR scanner.

9. The method of claim 8, wherein the imaging system employs a step of measuring proton-resonance frequency shift during the step of locating a heating effect of the subject's internal body tissue associated with the application of ultrasonic energy at the focal spot.

10. The method of claim 8, wherein the imaging system employs a step of measuring change of longitudinal relaxation time, T1, during the step of locating a heating effect of the subject's internal body tissue associated with the application of ultrasonic energy at the focal spot.

11. The method of claim 8, wherein the imaging system employs a step of measuring change in net polarization during the step of locating a heating effect of the subject's internal body tissue associated with the application of ultrasonic energy at the focal spot.

12. The method of claim 7, wherein the locating step includes applying energy from the focused ultrasound transducer at a lower power than during the step of debulking visceral fat.

13. The method of claim 6, further comprising a step of moving the focal spot of the focused ultrasound transducer with respect to the at least one target region containing visceral fat within the subject after or during the locating step.

14. The method of claim 13, wherein the moving step includes physically moving the focused ultrasound transducer with respect to the subject.

15. The method of claim 13, wherein the moving step includes at least one of the steps of changing a relative amplitude of a drive signal of the focused ultrasound transducer and changing a relative phase of the drive signal of focused ultrasound transducer.

16. The method of claim 6, wherein the imaging system includes an ultrasound scanner.

17. The method of claim 6, wherein the imaging system is includes x-ray scanner.

18. The method of claim 1, further comprising a step of monitoring application of ultrasonic power during or after the step of debulking visceral fat using an internal body tissue imaging system.

19. The method of claim 18, wherein the monitoring step includes using the internal body tissue imaging system to measure a heating effect of the subject's internal body tissue associated with the application of ultrasonic energy at the focal spot during or after the step of debulking visceral fat.

20. The method of claim 19, wherein the imaging system is an MR scanner.

21. The method of claim 20, wherein the imaging system employs a step of measuring proton-resonance frequency shift during the step of measuring a heating effect of the subject's internal body tissue associated with the step of debulking visceral fat.

22. The method of claim 20, wherein the imaging system employs a step of measuring change of longitudinal relaxation time, T1, during the step of measuring a heating effect of the subject's internal body tissue associated with the step of debulking visceral fat.

23. The method of claim 20, wherein the imaging system employs a step of measuring change in net polarization during the step of measuring a heating effect of the subject's internal body tissue associated with the step of debulking visceral fat.

24. The method of claim 1, wherein the positioning step includes a step of acoustically coupling the focused ultrasound transducer with respect to the subject.

25. The method of claim 1, wherein the step of debulking visceral fat within the target region includes a step of slewing the focal spot within the target region.

26. The method of claim 25, wherein the slewing step includes at least one of changing the shape of the transducer, employing an acoustic lens and applying selected amplitudes and phases to the elements of the multi-element transducer.

27. The method of claim 1, [[2, 3, 4, 5, 6, 7, 8, 9, 10, wherein the step of debulking visceral fat within the target region includes applying an ultrasound energy level and at a time duration suitable for debulking visceral fat by thermal necrosis.

28. The method of claim 1, wherein the step of debulking visceral fat within the target region includes applying an ultrasound energy level and at a time duration suitable for cavitation.

29. The method of claim 6, wherein the locating step includes using Acoustic Radiation Force Imaging to locate the focal spot.

30. A system for debulking visceral fat in a subject, the system comprising:
a focused ultrasound transducer configured to deliver sufficient ultrasonic energy to kill tissue within a focal spot;
an internal body imaging system configured to locate a heating effect of the subject's internal body tissue associated with the application of ultrasonic energy at the focal spot;
an internal body imaging system controller containing appropriate controls and components to operate the internal body imaging system and configured to process the images and other data obtained by the internal imaging system;
a device controller in communication with the focused ultrasound transducer and the internal body imaging system controller; configured to locate the focal spot to a region of visceral fat within the subject.

31. The system of claim 30, wherein the focused ultrasound transducer is a piezoelectric device.

32. The system of claim 30, wherein the focused ultrasound transducer is a Capacitive Micromachined Ultrasound Transducer (CMUT).

33. The system of claim 30, further comprising an acoustic coupling between the ultrasound transducer and the subject.

34. The system of claim 33 wherein the acoustic coupling is comprised of a selected material having an acoustic impedance substantially similar to that of the subject.

35. The system of claim 30, wherein the imaging system is an MR scanner.

36. The system of claim 35, wherein the imaging system employs a Proton Resonance Frequency pulse sequence to measure temperature changes.

37. The system of claim 35, wherein the imaging system employs a pulse sequence sensitive to changes in T1 arising from temperature changes.

38. The system of claim 35, wherein the imaging system employs a pulse sequence sensitive to changes in polarization arising from temperature changes.

39. The system of claim 30, wherein the imaging system is an ultrasound scanner.

40. The system of claim 30, wherein the imaging system is an x-ray scanner.

41. The system of claim 30, wherein the device controller is configured to control an ultrasound energy level of the ultrasound transducer and time duration suitable for debulking visceral fat by thermal necrosis.

42. The system of claim 30, wherein the device controller is configured to control an ultrasound energy level of the ultrasound transducer and time duration suitable for debulking visceral fat by cavitation.

43. The system of claim 31, wherein the system employs a focused ultrasound transducer comprised of a single piezoelectric crystal.

44. The system of claim 32, wherein the system employs a focused ultrasound transducer comprised of plurality of elements, each driven with a selected amplitude and phase.

45. The system of claim 44, wherein the device controller is configured to vary the amplitude and phase of the drive signals used to drive the plurality of elements to move the focal spot without moving the focused ultrasound transducer with respect to the subject.

46. The system of claim 30, further comprising an actuator configured to move the focal spot during the application of ultrasound power under control of the device controller.

47. The system of claim 30, wherein the device controller is configured to control the focused ultrasound transducer.

48. The system of claim 30 wherein the device controller is configured to monitor the efficacy of the focused ultrasound transducer.

49. The system of claim 46, wherein the device controller is configured to cause the internal body imaging system controller to acquire temperature-sensitive images of the region of interest in the subject, before, during, and after the use of the focused ultrasound transducer.

50. The system of claim 30, wherein the internal imaging system is configured to measure proton-resonance frequency shift during the step of locating a heating effect of the subject's internal body tissue associated with the application of the ultrasonic energy at the focal spot.

51. The system of claim 30, wherein the internal imaging system is configured to measure change of longitudinal relaxation time, T1, during the step of locating a heating effect of the subject's internal body tissue associated with the application of the ultrasonic energy at the focal spot.

52. The system of claim 30, wherein the internal imaging system is configured to measure change in net polarization during the step of locating a heating effect of the subject's internal body tissue associated with the application of the ultrasonic energy at the focal spot
